(19) United States
(12) Patent Application Publication
(10) Pub. No.: US 2026/0151535 A1
(43) Pub. Date: Jun. 4, 2026

(54) AMNIOTIC MEMBRANE DRESSING WITH IMPROVED TRANSPARENCY AND METHOD OF MANUFACTURING THE SAME

(71) Applicant: MSBIO, Inc., Seongnam-si (KR)

(72) Inventors: SUNG PO KIM, Cheongju-si (KR); SAM HYUN JUNG, Cheongju-si (KR); SUNG HYUN KIM, Yongin-si (KR); WAN KI PARK, Gwangju-si (KR); MI HEE PARK, Daegu (KR)

(21) Appl. No.: 19/408,080

(22) Filed: Dec. 3, 2025

(30) Foreign Application Priority Data
Dec. 3, 2024 (KR) 10-2024-0177283

(51) Int. Cl. A61L 27/36; A61L 27/56

(52) U.S. Cl. CPC A61L 27/3604; A61L 27/3691; A61L 27/56; A61L 2430/16

(57) ABSTRACT

The present disclosure relates to an amniotic membrane dressing including an amniotic membrane that is treated with ethylene oxide gas and a method of manufacturing the same. The amniotic membrane dressing of the present disclosure has an effect of improving transparency and structural stability of the amniotic membrane dressing by reacting the amniotic membrane with the ethylene oxide gas.

### CROSS-REFERENCE TO RELATED PATENT APPLICATION

This application claims the benefit of Korean Patent Application No. 10-2024-0177283, filed on Dec. 3, 2024, in the Korean Intellectual Property Office, the disclosure of which is incorporated herein in its entirety by reference.

### TECHNICAL FIELD

The present disclosure relates to an amniotic membrane dressing with improved transparency and a method of manufacturing the same, and more specifically, to an amniotic membrane dressing with a multilayered structure of which transparency and structural stability are improved so as to be used for treatment of ocular surface diseases and a method of manufacturing the same.

### BACKGROUND

An amniotic membrane is a membrane that has a thickness of approximately 0.02 to 0.5 mm, surrounds amniotic fluid, and is positioned at an innermost part of a placenta. The amniotic membrane is avascular tissue that does not represent histocompatibility antigen, so that there is no rejection even when it is transplanted. In addition, it is known that the amniotic membrane serves to promote epithelialization during a wound healing process and inhibit scar formation at a wound site due to various components of the amniotic membrane.

A conventional amniotic membrane dressing had a problem that maintaining a shape thereof during hydration is difficult due to lack of structural stability and opacity due to a chemical crosslinking when stacking the amniotic membrane is increased.

Accordingly, as a result of efforts to overcome disadvantages of the conventional amniotic membrane dressing, the present inventors have solved the problems of the related art and developed an amniotic membrane dressing that has an excellent structural stability and improved transparency.

### SUMMARY

### Technical Problem

Various embodiments described in the present specification are directed to providing an amniotic membrane dressing which has excellent structural stability so that a shape thereof is well maintained without distortion during hydration and has improved transparency in order to solve the above-described problem.

The problems to be solved by the present disclosure are not limited to the above-described problems, and other problems not mentioned will be clearly understood by those skilled in the art from the description below.

### Technical Solution

In an embodiment, an amniotic membrane dressing with excellent transparency including an amniotic membrane that is treated with ethylene oxide gas may be provided.

Here, a visible light transmittance of the amniotic membrane dressing may be 87 to 95%.

Here, the treatment may be performed at a temperature of 30° C. to 60° C.

Here, the amniotic membrane may be further treated with physical crosslinking.

Here, the amniotic membrane dressing may have improved enzyme degradation resistance of the amniotic membrane.

Here, the amniotic membrane dressing may be for ophthalmic use.

Here, when worn on an eye, the amniotic membrane dressing may have one or more holes with a diameter of 100 to 1,000 m formed around a central portion of cornea.

According to another embodiment, a method of manufacturing an amniotic membrane dressing including:
1) positioning an amniotic membrane on a convex-shaped mold;
2) treating the amniotic membrane of the step 1) with ethylene oxide gas;
3) cutting the amniotic membrane treated in the step 2); and
4) separating the cut amniotic membrane of the step 3) from the mold to obtain a lens-shaped amniotic membrane may be provided.

Here, the convex-shaped mold in the step 1) may be adjusted to a curvature of ocular cornea.

Here, the treatment in the step 2) may be performed at a temperature of 30° C. to 60° C.

Here, the step 1) may further include performing a physical crosslinking treatment on the amniotic membrane.

Here, the physical crosslinking treatment may be an ultraviolet light irradiation or dehydrothermal treatment.

Here, wherein the step 3) may further include cutting the amniotic membrane so that one or more holes with a diameter of 100 to 1,000 m are formed around a central portion of cornea when the amniotic membrane dressing is worn on an eye.

Here, the visible light transmittance of the amniotic membrane obtained in the step 4) may be 87 to 95%.

### Advantageous Effects

An amniotic membrane dressing of the present disclosure has excellent structural stability, thereby well maintaining a shape thereof without distortion during hydration and is easily placed on an eyeball without being displaced from a central position of the eyeball, and thus a surgery for fixation or attachment of an amniotic membrane is not needed and irritation and discomfort resulting from the surgery can be greatly reduced. In addition, transparency is increased by increasing a visible light transmittance and increasing a degradation period of the amniotic membrane as enzyme degradation resistance is improved, so that the amniotic membrane dressing can be effectively used for treatment of ocular surface diseases.

The effects according to the present disclosure are not limited to the above-described effects, and other effects not mentioned will be clearly understood by those skilled in the art from the description below.

### BRIEF DESCRIPTION OF THE DRAWINGS

FIG. 1 is a front view of a mold used in manufacturing of an amniotic membrane dressing of the present disclosure.

FIG. 2 is a schematic view of an amniotic membrane dressing according to an embodiment of the present disclosure.

FIG. 3 is a photograph of an amniotic membrane dressing according to an embodiment of the present disclosure.

FIG. 4 is a view showing an experimental result comparing resistance to collagenase degradation of an amniotic membrane dressing manufactured according to an embodiment of the present disclosure.

FIG. 5 is a view showing an experimental result comparing transparency during hydration of an amniotic membrane dressing manufactured according to an embodiment of the present disclosure.

FIG. 6 is a graph showing an experimental result comparing a visible light transmittance of an amniotic membrane dressing manufactured according to an embodiment of the present disclosure. *p<0.05

FIG. 7 is a graph showing an experimental result comparing cytotoxicity of an amniotic membrane dressing manufactured according to an embodiment of the present disclosure.

FIG. 8 is a view showing a result of histological analysis (H&E staining) of an amniotic membrane dressing according to an embodiment of the present disclosure.

FIG. 9 is a comparative photograph taken immediately after an amniotic membrane dressing manufactured according to an embodiment of the present disclosure was worn on an eye together with a therapeutic lens.

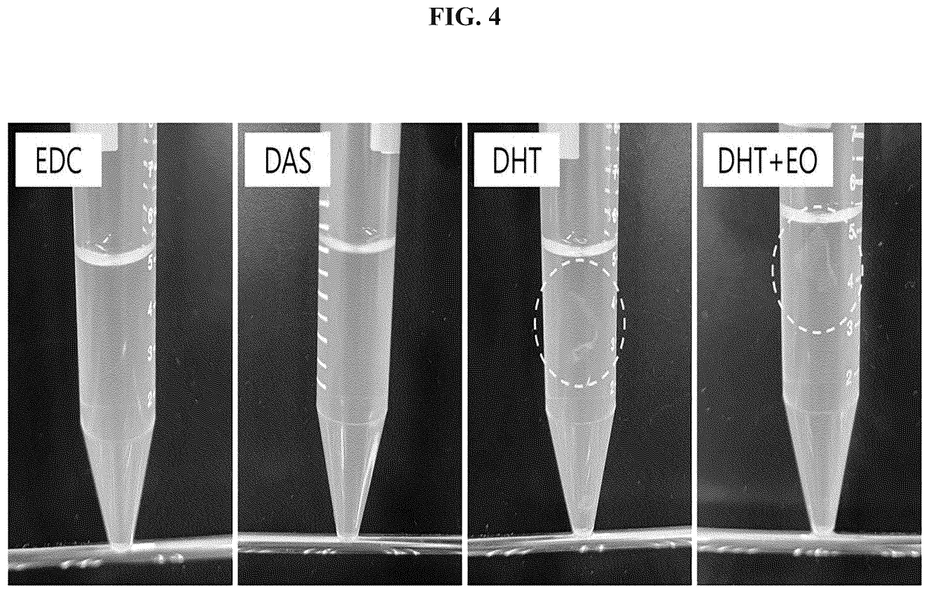

In FIGS. 4, 5, 6, 7 and 9, EDC represents Comparative Example 1, DAS represents Comparative Example 2, DHT represents Comparative Example 3, and DHT+EO represents Example 1.

### DETAILED DESCRIPTION OF THE EMBODIMENTS

The present disclosure may provide an amniotic membrane dressing including an amniotic membrane that is treated with ethylene oxide gas according to an embodiment.

The present disclosure may be characterized in that the amniotic membrane is treated with the ethylene oxide gas so as to improve transparency and stability of the amniotic membrane dressing. Here, the transparency may refer to transparency resulting from improvement of enzyme degradation resistance and/or a visible light transmittance. Here, the stability may refer to structural stability, and specifically, may refer to structural stability resulting from the improvement of enzyme degradation resistance and/or a condensation reaction.

In the present specification, the term “amniotic membrane dressing” may refer to one of sanitary materials manufactured using an amniotic membrane as a material, and in particular, may refer to a material that comes into contact with cornea for a purpose of treating ocular surface diseases.

The amniotic membrane may be an amniotic membrane derived from a mammal, and preferably, an amniotic membrane derived from a human or bovine source. The amniotic membrane may be a washed amniotic membrane or a decellularized amniotic membrane, and the amniotic membrane may be obtained directly from an excised placenta. Specifically, the amniotic membrane may be an amniotic membrane obtained by separating the amniotic membrane from a chorionic membrane of the placenta to remove residual tissue and blood clots and washing it. In addition, the amniotic membrane may be an amniotic membrane in which amniotic cells are removed by utilizing decellularization techniques known in the relevant field to eliminate potential risks of inflammatory or immune responses caused by the amniotic cells.

The amniotic membrane is composed of an epithelial layer, a basement membrane, a compact layer, a fibroblast layer, and a sponge layer and is also classified such that the compact layer, the fibroblast layer, and the sponge layer are included as an avascular mesenchymal stroma (stromal layer). The basement membrane of the amniotic membrane is composed of type III collagen, type IV collagen, type V collagen, laminin, and α6/β4 integrin, etc., and the stroma may mainly include type I collagen, type III collagen, hyaluronic acid, fibronectin, proteoglycan, etc. Collagen is composed of a repeating peptide chain with glycine(Gly)-X—Y-type tripeptide as one unit, and three peptide chains form a triple helix structure. Protease such as collagenase and trypsin may gradually degrade collagen and proteins by cleaving the interconnected peptide chains.

The amniotic membrane may be in a refrigerated, frozen, or dried state.

The amniotic membrane may be one layer or two or more layers. When overlapping two or more layers of the amniotic membrane on a mold, a thickness and strength of the amniotic membrane may be increased to achieve structural stability.

The quality of the amniotic membrane dressing may be determined by the thickness and strength of the amniotic membrane.

In the present specification, the term ethylene oxide (EO) may be used interchangeably with “oxidized ethylene”, and is a type of ether with a molecular formula of C₂H₄O.

The polypeptide present in collagen molecules constituting the amniotic membrane may include an amino terminus (N-terminus) having a free amino group and a carboxyl terminus (C-terminus) having a free carboxyl group. Among these, a free amine group of an amino acid is present in the polypeptide chain of the amino terminus, and among the amino acids constituting collagen, lysine or hydroxylysine may also have an amine group in a branched-chain. When a temperature of a tissue rises above body temperature, the collagen and proteins constituting the tissue change a three-dimensional structure to temporarily be in an unstable state, thereby exposing a reaction site that is not exposed at room temperature.

Accordingly, a chemical reaction between epoxide groups of ethylene oxide and the amine groups is actively carried out at a temperature of 30° C. to 60° C. and as a result, hydroxyethyl groups (N-2-hydroxyethyl groups) are introduced to form a more stabilized structure compared to a conventional crosslinking method. In addition, the hydroxyethyl groups introduced after the chemical reaction between the epoxide groups and the amine groups may inhibit an interaction between protease and peptide, thereby improving the enzymatic degradation resistance.

According to an embodiment, in the amniotic membrane treated with the ethylene oxide gas undergoes the condensation reaction in which the amine groups of collagen reacts with the epoxide groups of ethylene oxide, thereby improving transparency and structural stability and sufficiently reducing cytotoxicity compared to an amniotic membrane treated with conventional crosslinking.

The treatment may be performed at a temperature of 30° C. to 60° C. For example, the treatment may be performed at a temperature of 30° C. to 60° C., 30° C. to 55° C., 30° C. to 50° C., 30° C. to 45° C., 30° C. to 40° C., 30° C. to 35° C., 35° C. to 60° C., 35° C. to 55° C., 35° C. to 50° C., 35° C. to 45° C., 35° C. to 40° C., 40° C. to 60° C., 40° C. to 55° C., 40° C. to 50° C., 40° C. to 45° C., 45° C. to 60° C., 45° C. to 55° C., 45° C. to 50° C., 50° C. to 60° C., 50° C. to 55° C., or 55° C. to 60° C. When the temperature deviates from the above range, the chemical reaction between the epoxide groups of ethylene oxide and the amine groups may not occur actively, and thus a desired level of the structural stability and enzyme degradation resistance may not be achieved. In addition, when the temperature exceeds the above range, there may be a problem that the transparency of the amniotic membrane dressing is decreased due to a change in a structure of the protein of the amniotic membrane.

Here, a reaction time may be 1 to 6 hours, for example, 1 to 6 hours, 3 to 6 hours, 5 to 6 hours, 1 to 4 hours, or 3 to 4 hours. When the reaction time deviates from the above range, sufficient reaction may not occur, and thus the desired level of the structural stability and enzyme degradation resistance may not be achieved.

The visible light transmittance of the amniotic membrane dressing may be 87 to 95%. For example, the visible light transmittance may be 87 to 95%, 88 to 95%, 89 to 95%, or 90 to 95%. When the amniotic membrane is not treated with the ethylene oxide gas, the visible light transmittance may not reach the range.

The amniotic membrane may be further treated with chemical crosslinking or physical crosslinking. Specifically, the physical crosslinking may have been further treated before the amniotic membrane is treated with the ethylene oxide gas.

The physical crosslinking may be performed by ultraviolet light irradiation or dehydrothermal treatment (DHT).

The ultraviolet light irradiation may be performed by irradiating ultraviolet light in a wavelength of 254 to 305 nm on the amniotic membrane for 10 to 240 minutes.

The dehydrothermal treatment may be performed at a temperature of 45 to 105° C. and a pressure state (vacuum) of 0.1 to 100 mTorr for 24 to 168 hours. Specifically, the dehydrothermal treatment may be performed at a temperature of 45 to 105° C., 45 to 90° C., 45 to 80° C., 45 to 70° C., 45 to 60° C., 60 to 105° C., 60 to 90° C., 60 to 80° C., 60 to 70° C., 45 to 60° C., 80 to 105° C., 80 to 90° C., or 90 to 105° C.

Specifically, the dehydrothermal treatment may be performed for 24 to 168 hours, 24 to 140 hours, 24 to 120 hours, 24 to 100 hours, 24 to 80 hours, 24 to 60 hours, 24 to 40 hours, 50 to 168 hours, 50 to 140 hours, 50 to 120 hours, 50 to 100 hours, 50 to 80 hours, 50 to 60 hours, 80 to 168 hours, 80 to 140 hours, 80 to 120 hours, or 80 to 100 hours.

When the temperature and the time deviates from the above temperature and time ranges, sufficient physical crosslinking may not be performed, and thus the transparency and the structural stability may not be sufficient, and the cytotoxicity may not be sufficiently reduced.

The chemical crosslinking may be performed using a solution of at least one of dialdehyde starch (DAS), carbodiimide, EDC (or EDAC; 1-ethyl-3-(3-dimethylaminopropyl) carbodiimide), glutaraldehyde, formaldehyde, hexamethylene diisocyanate (HMDI), dextran, and glucose.

The amniotic membrane dressing may be for ophthalmic use, and specifically, for prevention or treatment of ocular surface diseases, but as long as it is for wearing on an eye, it is not limited thereto. Here, the term “prevention” may refer to any act of inhibiting or delaying an onset of the ocular surface diseases by wearing the amniotic membrane dressing on the eye according to an aspect. The term “treatment” may refer to any act that improves or beneficially modifies the ocular surface diseases by wearing the amniotic membrane dressing on the eye according to an aspect.

The amniotic membrane dressing may have a circular hole formed around a central portion of cornea when worn on the eye, and in this case, a number of the holes may be one or two or more, and a size of the hole may be about 100 to 1,000 m.

According to an embodiment, when the amniotic membrane dressing is cut into a curved shape (e.g., lens shape), it maintains a shape thereof during hydration due to excellent structural stability and transparency is increased, thereby having an effect of securing excellent visual acuity.

According to another embodiment, a method of manufacturing an amniotic membrane dressing including:
1) positioning an amniotic membrane on a convex-shaped mold;
2) treating the amniotic membrane of the step 1) with ethylene oxide gas;
3) cutting the amniotic membrane treated in the step 2); and
4) separating the cut amniotic membrane of the step 3) from the mold to obtain a lens-shaped amniotic membrane may be provided.

Among the term elements mentioned in the method of manufacturing the amniotic membrane dressing, those that are identical to those already mentioned are as described above.

The step 1) may be positioning the amniotic membrane on a convex-shaped mold that is adjusted to a curvature of ocular cornea and then drying the same. Here, the mold may be composed of a curved portion in which the amniotic membrane is positioned and dried, a pillar portion supporting the curved portion, and a support portion supporting the pillar portion.

Here, the mold may be manufactured using a three-dimensional (3D) printable material, and preferably, may be manufactured using polydimethylsiloxane (PDMS) or polylactic acid (PLA). In addition, the mold may be made of acryl, acetal, polytetrafluoroethylene (Teflon), stainless steel or titanium that may be processed by injection or molding, but it is not limited thereto.

A size (e.g., diameter) and curvature radius of the mold may be appropriately adjusted depending on a patient and surgical purpose. For example, in order to manufacture an amniotic membrane suitable for nationality or ethnicity of the patient, reference literature and statistical data known in the relevant field may be utilized to reflect a size and curvature radius of cornea, and the mold may be designed accordingly.

In an embodiment, the amniotic membrane may be dried in the step 1). Here, the drying may be natural drying at room temperature or freeze drying.

In addition, the step 1) may further include performing a physical crosslinking treatment on the amniotic membrane.

The step 2) may be reacting the amniotic membrane that is dried in the step 1) with the ethylene oxide (EO) gas.

Here, a purification time may be 5 to 24 hours.

Here, a reaction may be performed in an order of preheating, adjustment, charging, EO gas reaction, exhaustion, and purification completion.

The step 3) may be cutting the amniotic membrane treated in the step 2) into a shape of a standardized lens.

Specifically, the cutting may be cutting the amniotic membrane positioned at a boundary between the curved portion and the pillar portion of the mold by perforating using a contact lens-shaped blade, or by a rotary cutting method.

The cutting and processing may be performed through a process such as rotary cutting processing using a lathe and laser, utilizing a conventional technique well known in the relevant field, but it is not limited thereto.

The step 4) may be separating the amniotic membrane that is cut in the step 3) from the mold to obtain a lens-shaped amniotic membrane.

Here, the separation may be separating by hydration.

A solution that may be used during hydration may be sterile distilled water, sterile saline, dulcecco's modified eagle medium, phenol red free (DMEM) that is a tissue culture medium, etc.

Here, storing may be further included after freezing, freeze-drying, natural drying, or dehydration drying. The freezing may be freeze storing at −80° C. by immersing the amniotic membrane in a cryopreservation solution in which glycerol and DMEM are mixed, and the dehydration drying may be drying by dehydrating at room temperature after hydration in 70% ethanol, but it is not limited thereto.

Here, sterilizing by gamma-ray irradiation or electron beam irradiation may be further included. Safety in use may be ensured through the step.

Hereinafter, preferred examples are presented to help understand the present disclosure. However, the following examples are provided only for better understanding of the present disclosure, and the content of the present disclosure is not limited by the following examples.

### Preparation Examples

### Preparation Example 1: Preparation of Amniotic Tissue

An amniotic membrane was processed and treated in compliance with regulations on human tissue safety in accordance with the Safety and Management of Human Tissue Act of Korea. Specifically, the amniotic membrane was separated from a chorionic membrane of a placenta obtained after cesarean section of a donor mother confirmed to be free from infectious diseases such as hepatitis B, hepatitis C, acquired immunodeficiency syndrome (AIDS, HIV), or syphilis, and residual tissue and blood clots were removed. Next, the amniotic membrane was washed repeatedly for multiple times in sterile saline and a sodium hypochlorite solution, and finally washed repeatedly in sterile saline. The washed amniotic membrane was trimmed to an appropriate size capable of manufacturing the amniotic membrane dressing of the present disclosure, placed in a cryopreservation solution in which DMEM and glycerol are mixed in a ratio of 4:1 (v/v), and stored in a freezer at −40° C. or less until use. The freeze stored amniotic membrane was thawed at room temperature before use and then immersed in the sterile saline to remove the cryopreservation solution.

### Preparation Example 2: Preparation of Mold

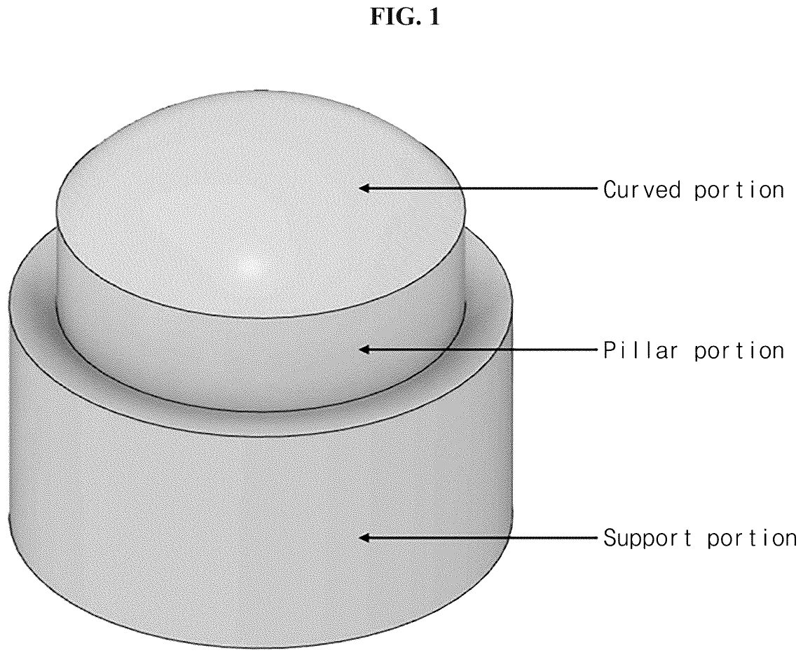

A mold was prepared in 7.8 mm to suit an average curvature radius of cornea of a Korean. A structure of the mold used is shown in FIG. 1. The mold may be composed of a curved portion in which the amniotic membrane is positioned and dried, a pillar portion supporting the curved portion, and a support portion supporting the pillar portion and is manufactured with polydimethylsiloxane (PDMS) or polylactic acid (PLA).

### Example 1

### 1. Drying of Amniotic Membrane

After placing an epithelial-cell side of the amniotic membrane prepared in Preparation Example 1 directly on the curved portion of the mold prepared in Preparation Example 2, it was air-dried at room temperature. Thereafter, it was placed thereon once more amniotic membrane and sufficiently air-dried.

### 2. Crosslinking of Amniotic Membrane

Next, an additional crosslinking process was performed so that the dried amniotic membrane forms a more stabilized structure. Specifically, dehydrothermal treatment was performed as a physical crosslinking method by treating the amniotic membrane at 45 to 60° C. in a vacuum state of 100 mTorr or less for 24 to 120 hours.

### 3. Ethylene Oxide Gas Reaction of Amniotic Membrane

Next, an active chemical reaction between epoxide groups of ethylene oxide (EO) and amine groups was induced so that the crosslinked amniotic membrane may form a more stabilized structure. Specifically, an EO gas sterilizer was used as a reaction equipment, and a disposable EO gas cartridge (Korea Chemical Engineering & Technology, CAPOX-100) was inserted into a cartridge holder inside the chamber, the crosslinked amniotic membrane was placed inside the chamber, and then the chamber door was closed and the EO gas reaction conditions were set and operated. A reaction temperature was set to 30 to 60° C., a reaction time was set to 1 to 3 hours, and a purification time was set to 10 to 24 hours. A cycle was proceeded in an order of preheating, conditioning, filling, EO gas reaction, exhaustion, and purification completion. An amniotic membrane dressing that had not been cut was obtained by this Example.

### 4. Cutting and Processing of Amniotic Membrane

Next, the amniotic membrane was cut and processed so as to form a standardized lens shape. Specifically, the amniotic membrane was cut and processed by perforating the amniotic membrane using a contact lens-shaped blade. In addition, by cutting two circular holes with a size of 100 to 1,000 m around a central portion of cornea of the amniotic membrane dressing, secretions such as tears and exudates may move smoothly to a wound site of the cornea covered by the amniotic membrane dressing during surgery, thereby preventing the wound site from drying out or secretions from accumulating.

### 5. Separation of Amniotic Membrane Dressing and Mold

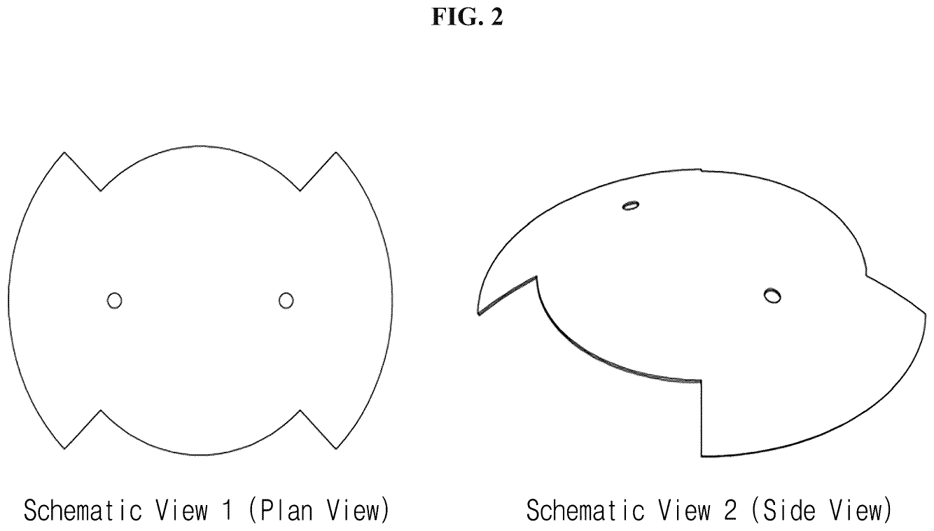

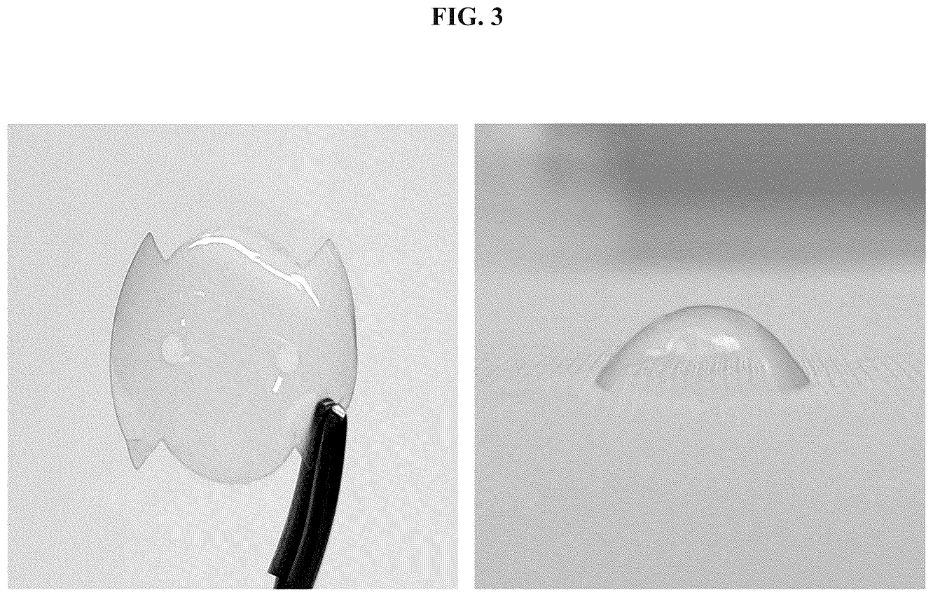

Next, after hydrating the amniotic membrane dressing having the standardized lens shape in sterile saline, and it was separated from the mold using a sterilized tweezer. The hydrated amniotic membrane dressing was immersed in 70% ethanol, and then recovered again and dehydrated at room temperature to dry. Next, a contact lens-type amniotic membrane dressing (Example 1) was obtained in a form shown in FIG. 2 and FIG. 3 by irradiating gamma rays at a dose of 15 to 25 kGy to sterilize.

### Comparative Examples

### Comparative Example 1: Manufacturing of Amniotic Membrane Dressing Treated with EDC

EDC (1-ethyl-3-(3-dimethylaminopropyl) carbodiimide) hydrochloride was used to manufacture an amniotic membrane dressing treated with chemical crosslinking.

Specifically, the dried amniotic membrane was immersed in a reagent in which a pH concentration was adjusted to 5 to 8 by mixing 1 to 10 mM of 2-morpholinoethanesulfonic acid (MES) in 40 to 70% aqueous ethanol solution, and stirred for 1 to 12 hours. Next, it was immersed in a reagent in which a pH concentration was adjusted to 5 to 8 by mixing 1 to 20 mM EDC hydrochloride and 1 to 10 mM N-hydroxysuccinimide (NHS) in 40 to 70% aqueous ethanol solution, and stirred for 1 to 12 hours. Next, the amniotic membrane was recovered, immersed in a 0.1 to 1.0 M sodium phosphate dibasic solution, stirred for 1 to 12 hours, removed, and then thoroughly washed with sterilized purified water and air-dried. Hereinafter, cutting and processing of the amniotic membrane and a separation process of the amniotic membrane dressing and the mold are performed in the same manner as in the Examples.

### Comparative Example 2: Manufacturing of Amniotic Membrane Dressing Treated with DAS

Dialdehyde starch (DAS) was used to manufacture the amniotic membrane dressing treated with chemical crosslinking.

Specifically, the dried amniotic membrane was immersed in a reagent in which a pH concentration was adjusted to 5 to 8 by mixing 10 to 100 mg/mL of dialdehyde starch in phosphate-buffered saline (PBS), and stirred for 1 to 60 minutes. Next, it was immersed in a 0.1 to 1.0% NaBH4/EtOH solution and stirred for 1 to 60 minutes. Next, the amniotic membrane was recovered, washed thoroughly with a reagent in which 0.1 to 1.0 M glycine is mixed in PBS, and air-dried. Hereinafter, cutting and processing of the amniotic membrane and a separation process of the amniotic membrane dressing and the mold are performed in the same manner as in the Examples.

### Comparative Example 3: Manufacturing of Amniotic Membrane Dressing Treated with DHT

Dehydrothermal treatment (DHT) was used to manufacture an amniotic membrane dressing treated with physical crosslinking.

Specifically, the dried amniotic membrane was treated at 45 to 60° C. in a vacuum state of 100 mTorr or less for 24 to 120 hours. Hereinafter, cutting and processing of the amniotic membrane and a separation process of the amniotic membrane dressing and the mold are performed in the same manner as in the Examples.

Accordingly, final samples obtained in Examples and Comparative Examples are as shown in Table 1 below.

TABLE 1

| Category | Sample name | Crosslinking method |
|---|---|---|
| Comparative | EDC | Used EDC hydrochloride |
| Example 1 | | as crosslinking reagent |
| Comparative | DAS | Used dialdehyde starch |
| Example 2 | | as crosslinking reagent |
| Comparative | DHT | Dehydrothermal treatment (DHT) |
| Example 3 | | |
| Example 1 | DHT + EO | Dehydrothermal treatment (DHT) + |
| | | Ethylene oxide gas reaction |

### Experimental Examples

### Experimental Example 1: Comparative Experiment on Resistance of Amniotic Membrane Dressing to Collagenase Degradation

In this Experimental Example, enzyme degradation resistance of an amniotic membrane dressing according to an embodiment of the present disclosure was confirmed.

Specifically, 2 mL of 0.1 M Tris-HCl (pH 7.4) solution including 0.05 M calcium chloride was added per 1 mg of specimen weight, and then allowed to stand at 37° C. for 1 hour. Next, after collagenase was manufactured in a 0.1 M Tris-HCl (pH 7.4) solution at a concentration of 200 Unit/mL, a same amount as the solution was added and stirred at 37° C. for 5 hours. The enzyme degradation resistance was evaluated by visually observing morphology of the specimen.

FIG. 4 is a view showing an experimental result comparing resistance to collagenase degradation of a contact lens-type amniotic membrane dressing manufactured according to an embodiment of the present disclosure.

As a result, referring to FIG. 4, morphology of the sample was not be confirmed in Comparative Examples 1 and 2. In Comparative Example 3, morphology of the sample that is not partially degraded was confirmed, but it was degraded to an extent that it was difficult to recognize that it is the amniotic membrane dressing. Meanwhile, in Example 1, a relatively intact sample was confirmed, and it was possible to recognize that it is the amniotic membrane dressing.

Therefore, it was confirmed that Example 1 is the most excellent in resistance of the amniotic membrane dressing to collagenase degradation compared to Comparative Examples in this experiment.

### Experimental Example 2: Comparative Experiment of Transparency During Hydration of Amniotic Membrane Dressing

In this Experimental Example, transparency during hydration of an amniotic membrane dressing was confirmed.

Specifically, the amniotic membrane dressing was hydrated in sterile saline contained in a 50 mm experimental culture dish, and then placed on a therapeutic lens to maintain a stable state. Thereafter, the amniotic membrane dressing was placed on a printed matter with specific characters printed thereon, and its transparency was evaluated by comparing legibility of the characters.

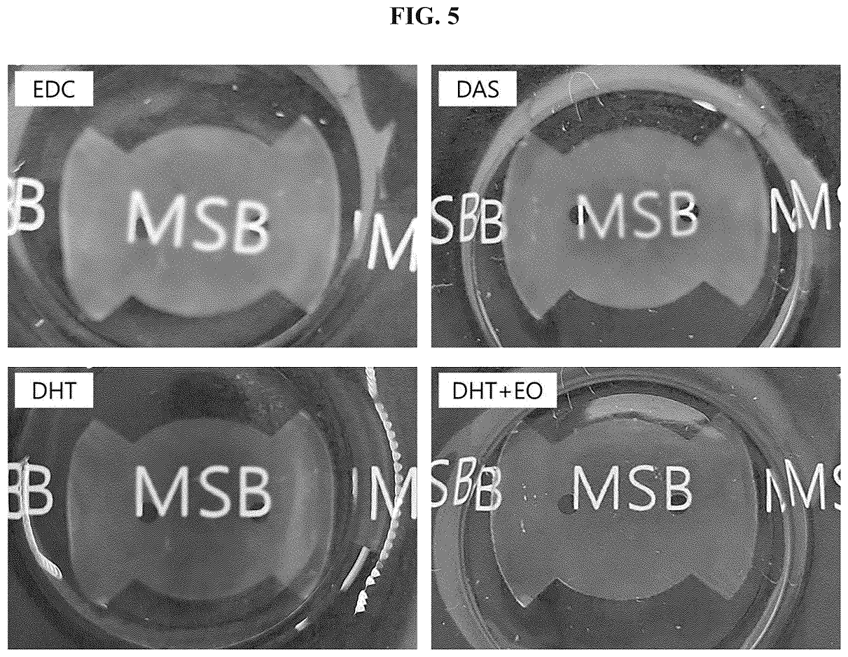

FIG. 5 is a view showing an experimental result comparing transparency during hydration of a contact lens-type amniotic membrane dressing manufactured according to an embodiment of the present disclosure.

As a result, referring to FIG. 5, the legibility of the characters decreased in an order of Comparative Example 3, Comparative Example 2, and Comparative Example 1. Example 1 exhibited the clearest visible characters. Therefore, it was confirmed that the transparency of Example 1 was the most excellent during the hydration of the amniotic membrane dressing compared to the Comparative Examples in this Experimental Example.

### Experimental Example 3: Comparative Experiment of Visible Light Transmittance of Amniotic Membrane Dressing

In this Experimental Example, an improvement effect of visible light transmittance of an amniotic membrane dressing was confirmed. Specifically, a UV-Visible Spectrophotometer (THERMO SCIENTIFIC, Evolution™ 201/220 UV/VIS Spectrophotometer) was used to measure the transmittance in a visible light region of 380 to 780 nm. Example 1 and Comparative Examples 1 to 3 were sufficiently hydrated in a cuvette for measuring a visible light transmittance containing sterile saline and positioned so as to be at a center of a light source portion, and then a light source in a form of a beam with a diameter of 6 mm was passed vertically through the amniotic membrane dressing in the cuvette to perform a measurement of the transmittance.

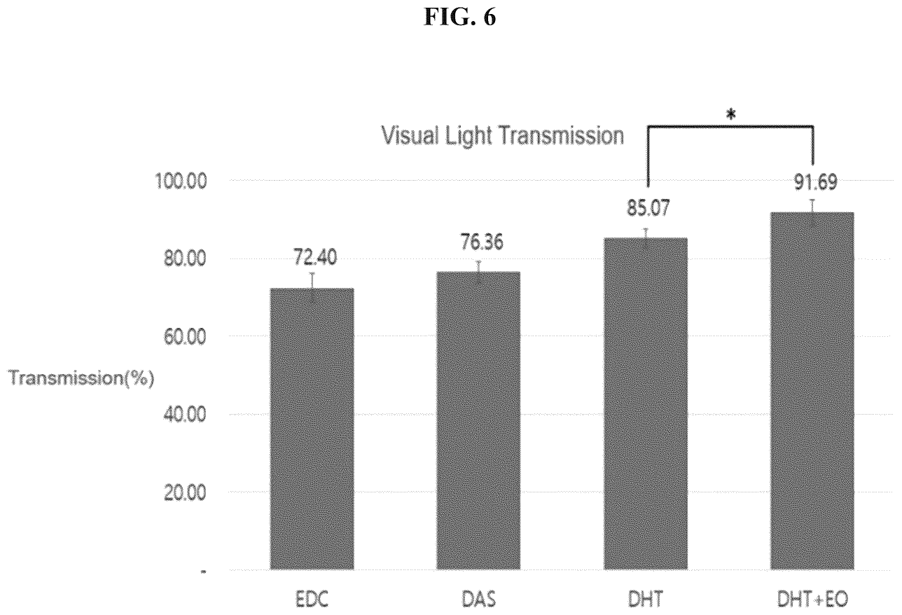

FIG. 6 is a graph showing an experimental result comparing a visible light transmittance of a contact lens-type amniotic membrane dressing manufactured according to an embodiment of the present disclosure.

As a result, referring to FIG. 6, a sample with the lowest visible light transmittance was Comparative Example 1, and a sample with the highest visible light transmittance was Example 1. In addition, it was confirmed that Example 1 showed a statistically significant improvement in the visible light transmittance compared to Comparative Example 3 that was not treated with ethylene oxide.

As a result of this Experimental Example, it was confirmed that the visible light transmittance of the amniotic membrane dressing was statistically significantly improved by ethylene oxide treatment.

### Experimental Example 4: Comparative Experiment of Cytotoxicity of Amniotic Membrane Dressing

In this Experimental Example, an improvement effect of cytotoxicity of an amniotic membrane dressing was confirmed. Specifically, a minimum essential medium (MEM) supplemented with 10% (v/v) fetal bovine serum (FBS) and 1% (v/v) penicillin-streptomycin was used as a culture medium. L-929 mouse fibroblast cells were used as a cell line. Extraction was performed by stirring at a rate of 1 mL of the culture medium per 6 cm² based on a surface area of each sample in a 5±1% CO₂ incubator at 37±1° C. for 72±2 hours with stirring, and then allowed to stand at room temperature for 1 hour, and a supernatant was collected and used as a test solution. In this Experimental Example, a medium without a sample was used as a control group, and a culture medium excluding the sample was used as the test solution. After the cell line was injected in a 1000 dish containing the MEM medium, a cell name, passage number, and date were recorded and cultured in the 5±1% CO₂ incubator at 37±1° C. for 72±2 hours, and the cells were dissociated using 1× Trypsin-EDTA twice a week and subcultured in a new culture medium. Monolayer cultured cells were treated with 1× Trypsin-EDTA to adjust a cell concentration to 1×10⁵ cells/mL, and 2 mL thereof was injected into a well (6-well tissue culture plate, Ø35 mm/well) of approximately 10 cm². After culturing for 24 to 48 hours in the 37±1° C., 5±1% CO₂ incubator, three or more monolayer cultured wells were selected for each condition and the medium was removed. 2 mL of the test solution was dispensed into the selected well according to each condition and cultured in the 37±1° C., 5±1% CO₂ incubator for 48±1 hours. After culturing, lysis or morphology of the cells was observed under a microscope.

Evaluation of cytotoxicity was determined according to cell viability according to the MTT cytotoxicity test. The MTT cytotoxicity test was referred to ISO 10993-5:2009, APPENDIX C. Specifically, an MTT solution was prepared by manufacturing MTT (3-(4,5-dimethylthiazol-2-yl)-2,5-diphenyltetrazolium bromide) at a concentration of 3 mg/mL in a PBS solution. The manufactured MTT solution was sterilized by aseptic filtration using a 0.2 m syringe filter. The MEM medium and the filtered MTT solution were homogeneously mixed at a ratio of 9:1, the culture medium was removed, 2 mL of the MTT solution was added to each well and then further cultured at 37±1° C. for 1.5 hours. Thereafter, the MTT solution was removed, 2 mL of DMSO (Dimethylsulfoxide) was added to each well, and the well plate was shaken to measure absorbance (570 nm) of the recovered solution.

Cell viability standard was set at 80% with reference to ISO 10993-5:2009 APPENDIX C.C.2.5 data.

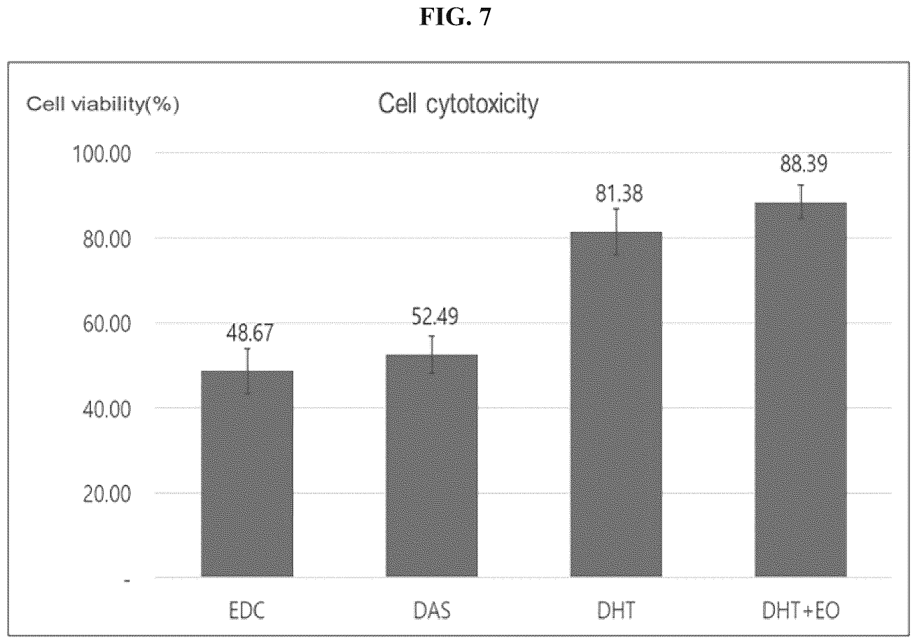

FIG. 7 is a graph showing an experimental result comparing cytotoxicity of a contact lens-type amniotic membrane dressing manufactured according to an embodiment of the present disclosure.

As a result, referring to FIG. 7, Comparative Example 1 and Comparative Example 2 did not reach the cell viability standard presented in this Experimental Example. On the other hand, Comparative Example 3 and Example 1 satisfied the cell viability standard and had no potential cytotoxicity.

This shows that a crosslinking reagent treated by a chemical method affected the cytotoxicity, and it was confirmed that a crosslinking method treated by a physical method was relatively safer than the chemical crosslinking method as the cytotoxicity was lower.

### Experimental Example 5: Histological Analysis Experiment of Amniotic Membrane Dressing

In this Experimental Example, tissue staining (H&E staining) was performed to confirm histological analysis of the amniotic membrane dressing of Example 1. Specifically, Example 1 was collected, fixed with a 10% formalin solution, washed and embedded in paraffin, then sectioned into a thickness of 5 m, stained with hematoxylin-eosin (H&E), and observed under an optical microscope.

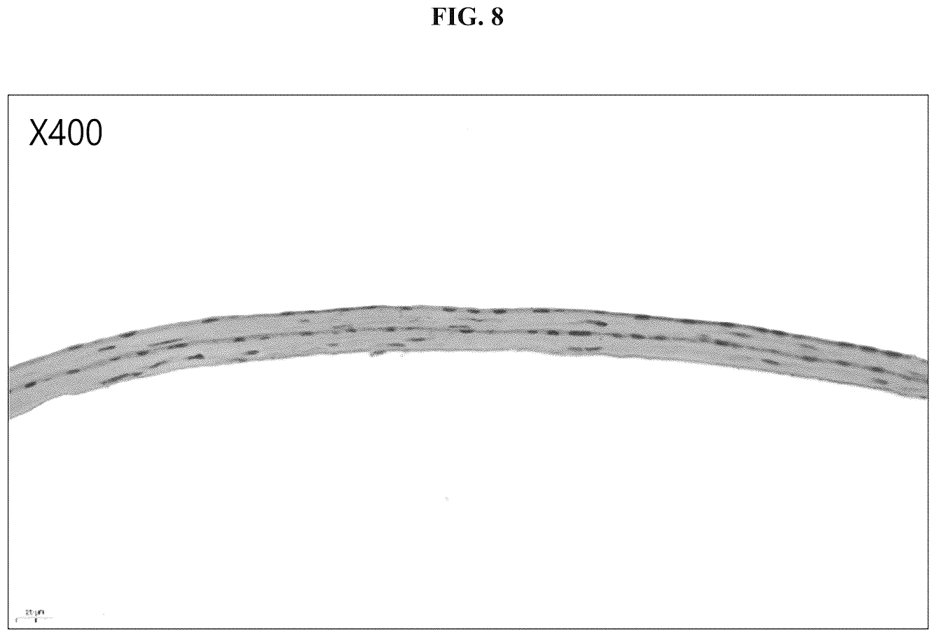

FIG. 8 is a view showing a result of histological analysis (H&E staining) of a contact lens-type amniotic membrane dressing according to an embodiment of the present disclosure.

As a result, referring to FIG. 8, it was confirmed that two layers of the amniotic membranes constituting the amniotic membrane dressing of Example 1 completely overlap each other, and the amniotic membrane dressing according to an embodiment of the present disclosure forms a stable multilayer structure. In each amniotic membrane, an epithelial layer and a basement membrane were observed, and below them, it was confirmed that it was composed of an avascular mesenchymal stroma in which fibroblasts were distributed around dense collagen fibers.

### Experimental Example 6: Comparative Experiment of Clinical Application Cases of Amniotic Membrane Dressing

In this Experimental Example, visual acuity and irritation according to clinical application of an amniotic membrane dressing was evaluated. Referring to the results of the transparency comparative experiment, the visible light transmittance comparative experiment, and the cytotoxicity comparative experiment of the Experimental Examples, Comparative Example 2 was set as a comparison group.

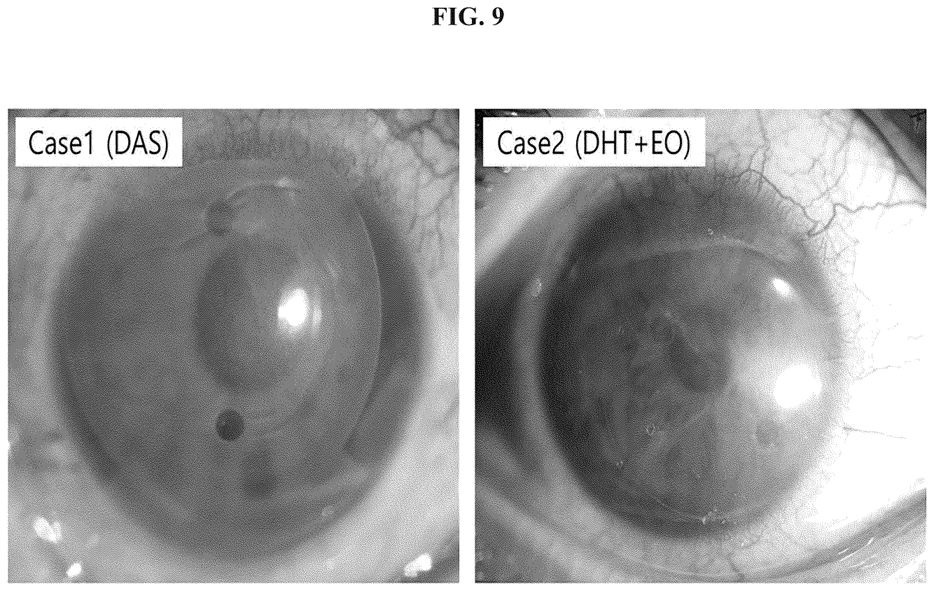

Specifically, a total of 16 general people aged 25 to 55 years with no ocular diseases and having normal visual acuity were selected as a subject for the clinical application experiment. After placing an amniotic membrane dressing hydrated in sterile saline on a therapeutic lens, a visual acuity test was performed using a visual acuity chart before and after wearing it on one eye, and an average value was recorded. Meanwhile, the subjects were asked to wear the amniotic membrane dressing for 1 hour and directly report the irritation they felt in 5 grades and for each grade, 1 point was given for minimal irritation, 2 points for slight irritation, 3 points for moderate irritation, 4 points for severe irritation, and 5 points for very severe irritation, and the average value was recorded. A photograph taken while wearing the amniotic membrane dressing in the clinical application case is shown in FIG. 9, and the result of the visual acuity test and irritation evaluation of the clinical application case is shown in Table 2 below.

TABLE 2

| | Case | | | |
|---|---|---|---|---|
| | Comparative Example 2 | | Example 1 | |
| | (DAS) | | (DHT + EO) | |
| Test(n = 8) | Before wear | After wear | Before wear | After wear |
| Visual acuity* | 0.76 ± 0.21 | 0.41 ± 0.19 | 0.75 ± 0.17 | 0.64 ± 0.18 |
| Irritation** | — | 4.13 ± 0.60 | — | 1.88 ± 0.78 |

*These visual acuity results were based on the Yong-Han, Jin chart.
**Irritation state: 1) Minimal: 1 2) Slight: 2 3) Moderate: 3 4) Severe: 4 5) Very Severe: 5

As a result, referring to Table 2, an average visual acuity of the subject of Comparative Example 2 was 0.76±0.21, and an average visual acuity of the subject of Example 1 was 0.75±0.17, showing that there was almost no difference in the average visual acuity between each subject. An average visual acuity of Comparative Example 2 measured after wearing was 0.41±0.19, whereas in a case of Example 1, an average visual acuity was 0.64±0.18, showing an improvement of about 0.23 compared to the result of Comparative Example 2. This may be determined as a result exhibited as transparency and visible light transmittance of the experimental group is more excellent compared to those of Comparative Example 2.

Thereafter, an average irritation score of Comparative Example 2 recorded after wearing the amniotic membrane dressing for 1 hour was 4.13±0.60, whereas an average irritation score of Example 1 was 1.88±0.78, which is about 2.25 lower than the result of Comparative Example 2. This may be determined as a result of the improved stability of the multilayer structure, which well maintains a shape thereof without distortion during hydration, is easily placed on an eye without being displaced from a central position of the eye, thereby reducing irritation, and Example 1 that was treated with crosslinking by the physical method had a lower cytotoxicity than Comparative Example 2 that was treated with crosslinking by the chemical method, and thus it may be determined that it is relatively safe from eye stimulation.

Taking the results together, the present disclosure may significantly reduce irritation and discomfort due to surgery as a shape thereof may be well maintained without distortion during hydration and is easily placed on an eye without being displaced from a central position of the eye by improving stability and transparency of a multilayer amniotic membrane dressing that may be used for treatment of ocular surface diseases, and may excellently serve as a contact lens-type amniotic membrane dressing in treatment of various ocular diseases, in particular, in treatment of a patient with cornea damage as increase in the transparency of the amniotic membrane dressing may be induced according to an increase in a visible light transmittance and increase in a degradation period of an amniotic membrane according to improvement of resistance to enzyme degradation.

The above-described contents are specific embodiments for carrying out the present disclosure. The present disclosure will include not only the above-described embodiments, but also embodiments that can be simply designed or easily modified. In addition, the present disclosure will also include techniques that can be easily modified and implemented using the above-described embodiments. Therefore, the scope of the present disclosure should not be limited to the above-described embodiments, but should be defined not only by the claims described below but also by equivalents of the claims of the present disclosure.

What is claimed is:

1. An amniotic membrane dressing with excellent transparency comprising an amniotic membrane that is treated with ethylene oxide gas.

2. The amniotic membrane dressing of claim 1, wherein a visible light transmittance of the amniotic membrane dressing is 87 to 95%.

3. The amniotic membrane dressing of claim 1, wherein the treatment is performed at a temperature of 30° C. to 60° C.

4. The amniotic membrane dressing of claim 1, wherein the amniotic membrane is further treated with physical crosslinking.

5. The amniotic membrane dressing of claim 1, wherein the amniotic membrane dressing has improved enzyme degradation resistance of the amniotic membrane.

6. The amniotic membrane dressing of claim 1, wherein the amniotic membrane dressing is for ophthalmic use.

7. The amniotic membrane dressing of claim 1, wherein, when worn on an eye, the amniotic membrane dressing has one or more holes with a diameter of 100 to 1,000 m formed around a central portion of cornea.

8. A method of manufacturing an amniotic membrane dressing, the method comprising:
1) positioning an amniotic membrane on a convex-shaped mold;
2) treating the amniotic membrane of the step 1) with ethylene oxide gas;
3) cutting the amniotic membrane treated in the step 2); and
4) separating the cut amniotic membrane of the step 3) from the mold to obtain a lens-shaped amniotic membrane.

9. The method of claim 8, wherein the convex-shaped mold in the step 1) is adjusted to a curvature of ocular cornea.

10. The method of claim 8, wherein the treatment in the step 2) is performed at a temperature of 30° C. to 60° C.

11. The method of claim 8, wherein the step 1) further includes performing a physical crosslinking treatment on the amniotic membrane.

12. The method of claim 11, wherein the physical crosslinking treatment is an ultraviolet light irradiation or dehydrothermal treatment.

13. The method of claim 8, wherein the step 3) further includes cutting the amniotic membrane so that one or more holes with a diameter of 100 to 1,000 m are formed around a central portion of cornea when the amniotic membrane dressing is worn on an eye.

14. The method of claim 8, wherein a visible light transmittance of the amniotic membrane obtained in the step 4) is 87 to 95%.
